(19) United States
(12) Patent Application Publication
(10) Pub. No.: US 2026/0137552 A1
(43) Pub. Date: May 21, 2026

(54) Facial Cold Plunge with Two Sections and Projections to Separate Solids

(71) Applicant: Nick Reed, Sheridan, WY (US)

(72) Inventor: Nick Reed, Sheridan, WY (US)

(21) Appl. No.: 19/295,619

(22) Filed: Aug. 10, 2025

Related U.S. Application Data
(63) Continuation-in-part of application No. 18/955,946, filed on Nov. 21, 2024.

(51) Int. Cl. A61F 7/00; C02F 1/00; C02F 1/28; C02F 103/02

(52) U.S. Cl. CPC A61F 7/0053; A61F 7/0085; C02F 1/001; C02F 1/283; A61F 2007/0003; A61F 2007/0063; A61F 2007/0069; A61F 2007/0086; C02F 2103/026

(57) ABSTRACT

A facial cold plunge device, comprising: a housing unit fabricated from thermally insulated material; an ergonomically contoured, bowl-shaped reservoir integrated into the housing unit; and a plurality of projections extending in a semicircular arc between two edges of the reservoir, wherein the projections define a central primary section configured to accommodate a user's face for water immersion on an interior aspect of the reservoir and a peripheral secondary section at the exterior aspect of the reservoir and wherein the projections are spaced closely enough to retain solids in the secondary section while allowing liquid to pass between the primary section and the secondary section.

### CROSS-REFERENCE TO RELATED APPLICATION

This application is a continuation in part of U.S. patent application Ser. No. 18/955,946 and claims the benefit of priority thereof.

### BACKGROUND OF THE INVENTION

The present invention relates generally to devices and systems for cold therapy, particularly to portable, compact apparatuses designed for facial immersion in cold water. More specifically, this invention pertains to a facial cold plunge device with integrated chilling, filtration, and a retractable breathing apparatus, enabling prolonged and convenient cold water immersion for therapeutic, cosmetic, and wellness applications. The present invention is designed to be placed in a newly manufactured or preexisting cold plunge tub.

### BRIEF SUMMARY OF THE INVENTION

The present invention provides a novel apparatus for facilitating cold water immersion therapy specifically targeting the face, to be placed in a newly manufactured or preexisting cold plunge tub.

An embodiment of the present invention is a one piece bowl that has two sections the water can flow through. The part of the top is for putting ice (as well as possibly lemon or cucumber) and the water flows through and so the water will be cold but the user will not have any ice stuck to their face.

It solves a major customer problem and that is getting ice stuck in their face or having lemon in their nose while they are plunging so it keeps all the matter out of the face bowl compartment.

### BRIEF DESCRIPTION OF THE FIGURES

FIG. 1: illustrates the key components of the facial cold plunge device configured to operate in conjunction with an existing cold plunge tub.

### DETAILED DESCRIPTION

The present invention is directed to a novel and advanced facial cold plunge device, for use in conjunction with a cold plunge tub. The invention comprises a main housing unit fabricated from thermally insulated materials, such as high-grade thermoplastics or stainless steel, designed to maintain the thermal integrity of the internal water reservoir.

The housing contains an ergonomically contoured, bowl-shaped reservoir specifically designed to conform to the user's facial profile, enhancing comfort and immersion efficiency while minimizing water spillage. Embedded beneath the reservoir may be optionally an integrated chilling mechanism employing a miniature compressor or thermoelectric cooling system. This system can incorporate evaporative cooling coils positioned strategically to ensure uniform cooling across the reservoir, maintaining water temperatures below 50° F.

The chilling mechanism can be controlled via an electronic thermostat interface, equipped with a digital display for real-time temperature readings and programmable settings, allowing the user to customize and maintain precise water temperatures for optimal therapeutic effect.

The present invention is designed to attach to the wall of a full-body cold plunge, enabling the integration of facial immersion therapy into an existing system. In this configuration, the device features an adaptable mounting mechanism that securely affixes the ergonomic reservoir to the rim or sidewall of a full-body plunge tub.

A submersible pump, operatively connected to the main unit, may draw water from the full-body plunge tub into the facial immersion bowl and recirculates it back into the tub. This closed-loop circulation system eliminates the need for separate chilling and filtration mechanisms within the facial device itself, as the water is maintained at the desired temperature and cleanliness by the full-body plunge's primary systems.

The device may further incorporate a filtration unit. The pump continuously circulates water through a filtration assembly, which includes replaceable carbon or HEPA filters to remove impurities, bacteria, and debris, ensuring the water remains clean and suitable for repeated use without requiring frequent draining or replacement.

The circulation system may also incorporate insulated conduits to prevent thermal losses during operation, ensuring consistent and efficient cooling. A unique feature of the invention is the retractable breathing apparatus, which allows users to sustain facial submersion for extended durations without the need to interrupt the immersion process to breathe. This apparatus comprises a snorkel-like breathing tube constructed of medical-grade silicone, which is mounted on the housing unit adjacent to the reservoir.

The breathing apparatus, ergonomic reservoir, and optional UV sterilization components ensure users experience the same therapeutic and cosmetic benefits while leveraging existing equipment.

This configuration enhances the versatility and cost-effectiveness of the invention, making it accessible to users who already utilize full-body cold plunge systems but desire the convenience and specificity of facial cold therapy.

### DETAILED DESCRIPTION OF FIGURES

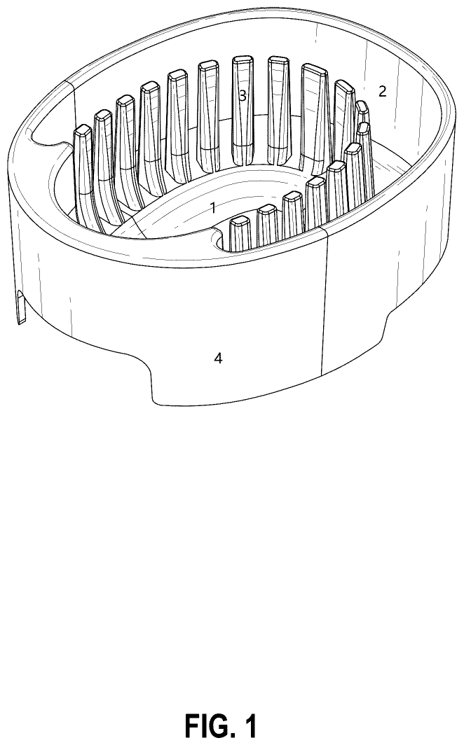

FIG. 1: illustrates the structural and functional components of the facial cold plunge device used in conjunction with a cold plunge tub. The following components are labeled with their respective reference numerals:
a housing unit fabricated from thermally insulated material 4;
a plurality of projections extending in a semicircular arc between two edges of the reservoir 3,
a central primary section 1 configured to accommodate a user's face for water immersion on an interior aspect of the reservoir and a peripheral secondary section 2 at the exterior aspect of the reservoir.

The projections are spaced closely enough to retain solids in the secondary section while allowing liquid to pass between the primary section and the secondary section.

### Optional Features

Integrated assembly of cold plunge tub and facial plunge device The adjustable thermostat, integrated into the device, allows users to precisely set and maintain the desired water temperature. The thermostat includes a digital interface displaying real-time temperature readings and programmable controls for custom settings.

### Cold Plunge Tub

The filtration system, identified as component, ensures water hygiene by removing impurities, bacteria, and debris. The filter is replaceable and part of the closed-loop water circulation system, maintaining the cleanliness of the water for multiple uses.

### Lowering Attached Breathing Mechanism

The attached breathing mechanism is a retractable snorkel-like tube that lowers into position for use. This component includes an adjustable height mechanism and a two-way check valve to allow seamless respiration while preventing water ingress during facial immersion.

### Telescoping Legs

Positioned adjacent to the filtration system, the UV light sterilizes the circulating water by eliminating microbial contaminants, further enhancing water purity and safety during prolonged immersion.

### Removable Mouthpiece for Easy Cleaning

The mouthpiece of the breathing apparatus, is detachable to facilitate thorough cleaning. Constructed from medical-grade silicone, it ensures hygiene and comfort during repeated usage.

### Removable Mouthpiece for Easy Cleaning

The mouthpiece of the breathing apparatus is detachable to facilitate thorough cleaning. Constructed from medical-grade silicone, it ensures hygiene and comfort during repeated usage.

What is claimed is:

1. A facial cold plunge device, comprising:
a) a housing unit fabricated from thermally insulated material;
b) an ergonomically contoured, bowl-shaped reservoir integrated into the housing unit; and
c) a plurality of projections extending in a semicircular arc between two edges of the reservoir, wherein the projections define a central primary section configured to accommodate a user's face for water immersion on an interior aspect of the reservoir and a peripheral secondary section at the exterior aspect of the reservoir and wherein the projections are spaced closely enough to retain solids in the secondary section while allowing liquid to pass between the primary section and the secondary section.

2. The facial cold plunge device of claim 1, wherein the reservoir is contoured to provide a watertight seal against the user's face to minimize water spillage during use.

3. The facial cold plunge device of claim 1, further comprising a chilling mechanism is operable to cool water within the reservoir to temperatures as low as 32° F. while maintaining uniform cooling across the reservoir surface.

4. The facial cold plunge device of claim 1, further comprising a filtration assembly comprises a carbon or HEPA filter capable of removing bacteria, debris, and other impurities from the water during operation.

5. The facial cold plunge device of claim 1, further comprising a retractable breathing apparatus is operatively mounted on a spring-loaded mechanism to allow stowing when not in use and deployment during immersion.

6. The facial cold plunge device of claim 1, wherein a two-way check valve of the breathing apparatus is configured to allow seamless inhalation and exhalation while submerged in water.

7. The facial cold plunge device of claim 1, wherein a thermostat control system includes a programmable timer function to automatically maintain the desired water temperature over a predefined period.

8. The facial cold plunge device of claim 1, wherein the housing unit comprises a compact, lightweight design with rubberized feet or suction cups for enhanced stability during operation.

9. The facial cold plunge device of claim 1, further comprising an optional battery-powered mode to enable portable and off-grid use.

10. The facial cold plunge device of claim 1, wherein a water circulation system is configured to operate continuously to maintain consistent water temperature and filtration during extended periods of use.

11. The facial cold plunge device of claim 1, wherein a breathing apparatus is adjustable in length and height to accommodate users of varying facial dimensions and preferences.

12. The facial cold plunge device of claim 1, wherein anergonomic reservoir is constructed with non-slip interior coatings to enhance user safety and comfort during immersion.

13. The facial cold plunge device of claim 1, whereinadigital display of the thermostat system provides real-time feedback on water temperature and system status.

14. The facial cold plunge device of claim 1, further comprising an integrated safety mechanism configured to shut down the chilling mechanism and pump in the event of water leakage or overheating.

15. The facial cold plunge device of claim 1, wherein the device is configured to support detachable accessories, including alternative filtration systems and enhanced breathing apparatus configurations.

16. A variation of the facial cold plunge device of claim 1, wherein the device is configured to attach to the wall of a full-body cold plunge system, comprising:
a) a mounting mechanism adapted to secure the reservoir to the rim or sidewall of the full-body cold plunge tub;
b) a submersible pump operatively connected to the reservoir, configured to draw water from the full-body plunge tub into the reservoir and return it, thereby utilizing the primary chilling and filtration systems of the full-body plunge; and
c) a closed-loop circulation system enabling continuous water flow between the full-body cold plunge tub and the reservoir, while maintaining ergonomic and respiratory functionalities of the facial cold plunge device.
